(19) United States
(12) Patent Application Publication
(10) Pub. No.: US 2015/0295178 A1
(43) Pub. Date: Oct. 15, 2015

(54) 3-SUBSTITUTED TELLUROPHENES AND RELATED COMPOUNDS

(71) Applicant: THE GOVERNING COUNCIL OF THE UNIVERSITY OF TORONTO, Toronto, Ontario (CA)

(72) Inventors: Dwight Seferos, Mississauga (CA); Ashlee Jahnke, Totonto (CA)

(21) Appl. No.: 14/440,305

(22) PCT Filed: Nov. 4, 2013
(86) PCT No.: PCT/CA2013/050837

§ 371 Date: May 1, 2015

Related U.S. Application Data
(60) Provisional application No. 61/721,758, filed on Nov. 2, 2012.

(51) Int. Cl. H01L 51/00; C08L 65/00; C08J 5/18; C07D 345/00; C08G 61/12

(52) U.S. Cl. CPC H01L 51/0035; C07D 345/00; C08G 61/123; C08J 5/18; C08L 65/00; C08J 2365/00; H01L 51/42

(57) ABSTRACT

Monomeric 3-substituted tellurophene compounds, as well as their use in the synthesis of oligomeric and/or polymeric compounds consisting of two or more tellurophene-2,5-diyl groups which are covalently linked to each other are disclosed, as is the use of said oligomers and polymers in devices such as diodes and solar cells, electrodes and semiconductors.

### RELATED APPLICATION

This application claims the benefit of U.S. Provisional Patent Application Ser. No. 61/721,758 filed Nov. 2, 2012.

### FIELD OF THE INVENTION

The invention relates to 3-substituted tellurophenes, polytellurophenes, and related compounds, methods of synthesis and use.

### BACKGROUND

Polythiophenes have been extensively studied and characterized, and are important organic electronic materials.¹ While furan² and selenophene³ analogs have emerged in recent years, there have been very few reports of soluble tellurophene-containing polymers.⁴

### SUMMARY

An embodiment of the invention is an oligomeric or polymeric compound containing two or more tellurophene-2,5-diyl groups covalently linked to each other, the covalent linkage between the monomeric groups being between ring carbons adjacent (directly bonded to) the Te atom. Such positions are numbered the 2- or 5-position of the tellurophene ring according to rules of nomenclature. Each tellurophene ring bears an R-group i.e., a monovalent organic radical at one of the 3- or 4-positions of the ring. Examples of monovalent organic groups are provided by the Examples described below, and thus include —CH₂CH₂CH₂CH₂CH₂CH₃, —CH₂—C(H)(CH₂HC₃)(CH₂CH₂CH₂CH₃), —(CH₂)₁₁CH₃ along with other monovalent organic radicals which when part of the compound have an atom covalently linked to a carbon atom of a tellurophene ring.

The invention includes oligomeric and polymeric compounds comprising a plurality of substituted tellurophene rings, as illustrated by formula (A) in which n is an integer greater than 1:

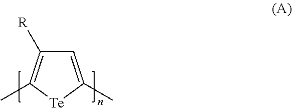

Oligomers are relatively small molecules in which n has a value of at least 2 and up to 10. The Mₙ of a polymer is at least 2000.

The invention thus includes compounds containing the structure shown by formula (A) in which n is an integer greater than 1.

Disclosed herein are compounds of formula (4) and formula (5):

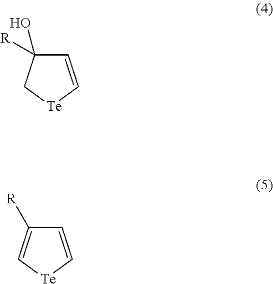

in which R is a monovalent organic substituent.

Also disclosed is compound of formula (B):

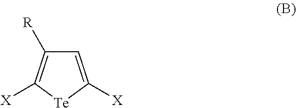

wherein:
   each X is, independently of the other X, F, Cl, Br, I, H, Li, Na, MgX¹, B(OR′)(OR″), or SnR′″₃, and if one X is H, then the other X is not H.

Compound (4) can be transformed into compound (5). Compound (5) can be transformed into compound (B). Molecules having formula (5) can be coupled to form polytellurophenes, and molecules having formula (B) can be coupled to form polytellurophenes.

In one embodiment, a polytellurophene is prepared by exposing a compound of formula (5) to an electrochemical potential of from 0.1 to 3.0 V.

Another embodiment includes preparing a polytellurophene by:
(i) activating a monomer of formula (B) at the 2 and/or 5 positions of the tellurophene ring; and
(ii) coupling or polymerizing activated monomers in the presence of a coordination catalyst.

A polymer of the invention can be useful when transformed into a film as, for example, as a part of a semiconductor composite material.

A method of the invention includes preparing a compound of formula (5) by dehydrating a compound of formula (4).

A further understanding of the functional and advantageous aspects of certain embodiments of the invention can be realized by reference to the following detailed description and drawings.

### BRIEF DESCRIPTION OF THE DRAWINGS

Embodiments will now be described, by way of example, reference being made to the accompanying drawings, in which:

FIG. 1 is a scheme showing the synthetic outline of 3-alkyltellurophenes.

FIG. 2 shows characterization of 3-hexyltellurophene by (a) cyclic voltammetry and (b) pulsed spectroelectrochemistry. All potentials are relative to Fc/Fc⁺.

FIG. 3 shows electrochemical polymerization of 3-hexyltellurophene.

FIG. 4 is a scheme showing the nickel-catalyzed polymerization of diiodo-alkyltellurophenes.

FIG. 5 provides solution absorption spectra of poly(3-alykyltellurophene) (left hand side) and representative proton NMR spectra (right hand side).

FIG. 6 provides normalized absorbance spectra of P3HTe in 1,2,4-trichlorobenzene at various temperatures from 25 to 95° C.

FIG. 7 shows (a) thin film absorption spectra of polymers P3EHTe, P3DDTe and P3HTe; (b) AFM image of P3EHTe spun cast onto glass substrates and annealed 1 h at 100° C., the inset showing the corresponding phase image; (c) cyclic voltammogram of P3HTe; and (d) SEC doping of P3HTe spun cast onto an ITO substrate. All potentials are relative to Fc/Fc⁺.

FIG. 8 shows an ¹H NMR spectrum of 3-hexyltellurophene (5a).

FIG. 9 shows an ¹H NMR spectrum of 2,5-diiodo-3-hexyltellurophene (6a).

FIG. 10 shows an ¹H NMR spectrum of 3-dodecyltellurophene (5b).

FIG. 11 shows an ¹H NMR spectrum of 2,5-diiodo-3-dodecyltellurophene (6b).

FIG. 12 shows an ¹H NMR spectrum of 3-(2′-ethylhexyl)tellurophene (5c).

FIG. 13 shows an ¹H NMR spectrum of 2,5-diiodo-3-(2′-ethylhexyl) tellurophene (6c).

FIG. 14 shows an ¹H NMR spectrum of poly(3-dodecyltellurophene) with asterisks indicating chloroform satellite peaks.

FIG. 15 shows an ¹H NMR spectrum of poly(3-(2′-ethyl)hexyltellurophene).

FIG. 16 shows calculated molecular orbitals of methyltellurophene pentamer and the predicted wavelengths of the two strongest transitions.⁵ The geometries of a five ring chain of 3-methyl tellurophene were optimized on the Gaussian 09 suit of programs⁶ using the nonlocal hybrid Becke three-parameter Lee-Yang-Parr (B3LYP) functional⁷ and the 6-31g(d) basis set for C and H atoms and LanL2DZ for Te (methyl groups in the 3-position of the thiophene were used in replace of hexyl chains to minimize computational time). The first twenty singlet excited-states were calculated with TD-DFT at the same level of theory and basis set used for the DFT calculations.⁸

FIG. 17 shows calculated absorbance spectrum for the methyltellurophene pentamer. The shown calculated UV-vis spectrum was generated from the TD-DFT data with Gausview by applying a gaussian function with 0.33 eV peak half width at half height placed on each transition.

FIG. 18 is a cyclic voltammogram of P3HTe thin film on an ITO substrate.

### DETAILED DESCRIPTION

As used herein, the terms, “comprises” and “comprising” are to be construed as being inclusive and open ended, and not exclusive. Specifically, when used in this specification including claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

Disclosed herein are the first known examples of substituted tellurophene homopolymers, which have been found to be soluble. There has been considerable debate as to the stability of these polymers in general. “Heavy” heterocycles offer certain advantageous properties relative to their lighter analogs, including a narrow optical band-gap,⁹ the ability to be polarized,¹⁰ the ability to form extended valence adducts,¹¹ enhanced planarity,^{3(e)} and a distinct solid-state structure.¹² The inventors believe that this is the first disclosure of 3-substituted tellurophenes and polymer prepared therefrom. Exemplifying the invention is a series of alkyltellurophene homopolymers, including the tellurium analog of the ubiquitous poly(3-hexylthiophene) (P3HT).

The polymers have been shown to have excellent stability. Exemplified polymers have been characterized in a demonstration of the feasibility of their use, for example, as an electronic material.

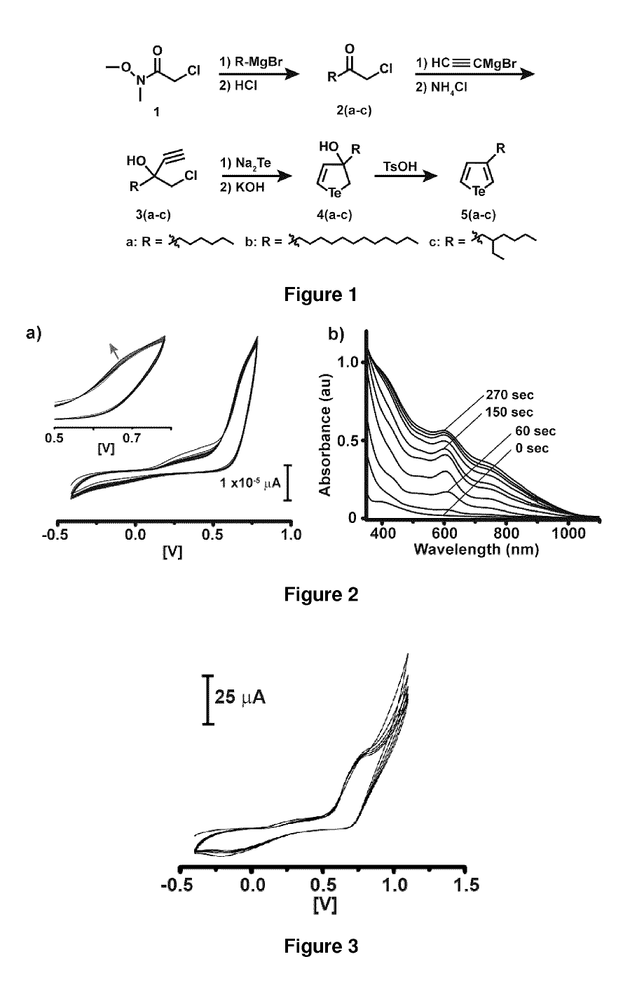

3-substituted tellurophene monomers were prepared by a ring closing reaction that places an alkyl substituent at the 3-position of the tellurophene ring, as shown in the scheme of FIG. 1.¹³ The exemplified synthesis begins with the preparation of the Weinreb amide 2-chloro-N-methoxy-N-methylacetamide (1).¹⁴ This precursor was then treated with hexylmagnesium bromide to afford 1-chloro-2-octanone (2a) in which the C—C linkage between the hexyl substituent and downstream tellurophene ring is formed. It was not found necessary to purify 2a. Treatment of 2a with ethynylmagnesium bromide affords 3-(chloromethyl)-1-nonyn-3-ol (3a), an upstream precursor to the five-member ring product. Addition of 3a to a solution of sodium telluride in ethanol gives the intermediate (4a), which was dehydrated without further purification to give 3-hexyltellurophene (5a). All three of the exemplary 3-alkyltellurophenes can be prepared in a similar manner. The ¹H NMR spectra of 3-hexyltellurophene (5a), 3-dodecyltellurophene (5b), and 3-(2′-ethylhexyl)tellurophene (5c) are shown in FIGS. 8, 10 and 12, respectively.

A hallmark of 3-alkylthiophenes is their ability to be electropolymerized at relatively low oxidative potential. Electrochemical polymerization of 3-hexyltellurophene (5a) was found to be possible. In the past, oxidative polymerization has been a common route to other polytellurophenes. This may be due to an inability to functionalize the tellurophene ring in the 2- and 5-positions, which is required for transition-metal catalyzed polymerization.

The electrochemical properties of 5a were investigated by cyclic voltammetry (CV) in acetonitrile, revealing two irreversible oxidation peaks at 0.56 V and 0.90 V (all potentials are reported vs. ferrocene/ferrocene⁺¹). Repeated CV cycling to 0.78 V did not produce a well-defined film on the surface of a platinum working electrode but a small increase in current and shift to lower oxidation potential was observed in the voltammogram indicating that electrochemical polymerization occurs to some extent (FIG. 2(a)). Similar results were obtained upon repeating the process and increasing the CV cycling range to 1.08 V (FIG. 3). When an electrochemical cell containing 5a was held at a constant potential (0.58 V) for a sustained period, however, the solution changed from colorless to blue and produced a blue precipitate. Based on this observation, spectroelectrochemical measurements were performed on 5a in dichloromethane to further investigate the electropolymerization process. Time-resolved spectroelectrochemical measurements were conducted using a platinum gauze working electrode that was held at a constant potential (0.58 V) for 30 second intervals, after which the absorbance profile was measured (FIG. 2(b)). Here, it was found that the absorbance in the visible region increases with each successive pulse, producing results consistent with the formation of poly(3-hexyltellurophene) (P3HTe). After 10 pulses the spectrum had an absorbance maximum at 599 nm with a well defined shoulder at around 750 nm. During the course of this experiment, trace amounts of an insoluble blue film were also noted to coat the surface of the working electrode. Overall, electrochemical and absorption spectroscopy measurements show that the electrochemical polymerization of 5a occurs.

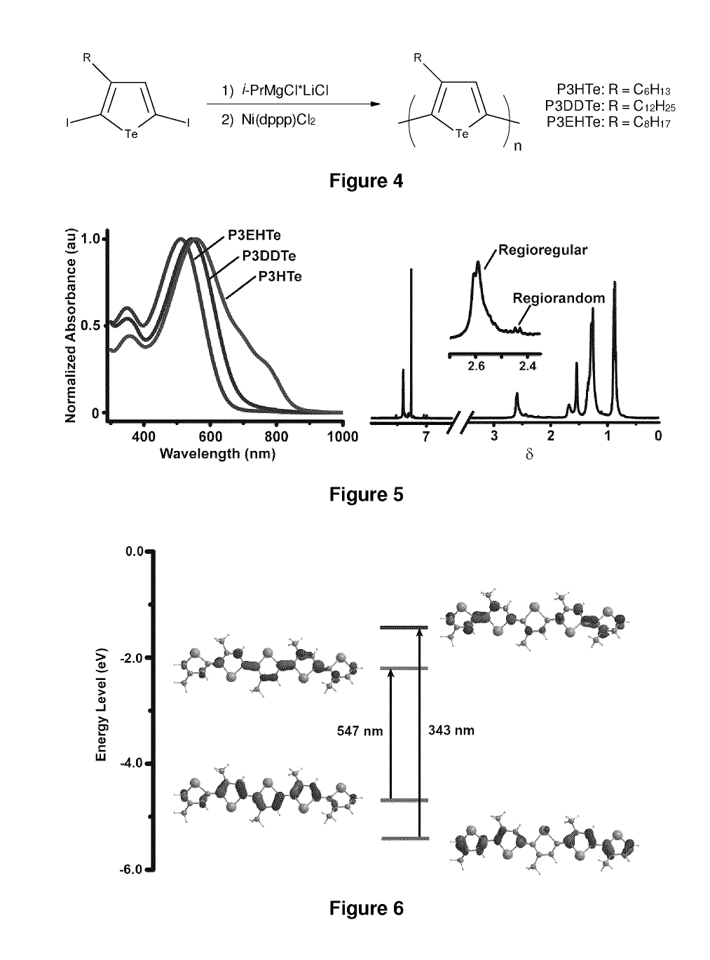

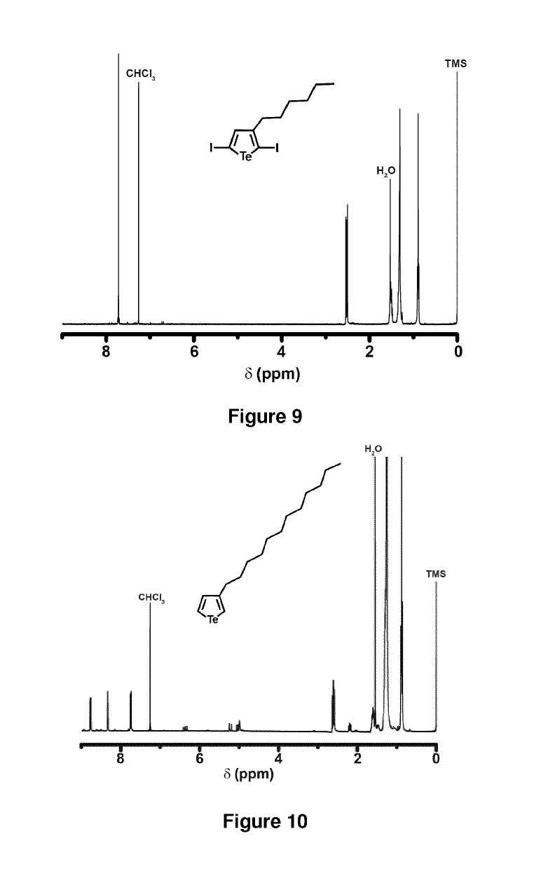

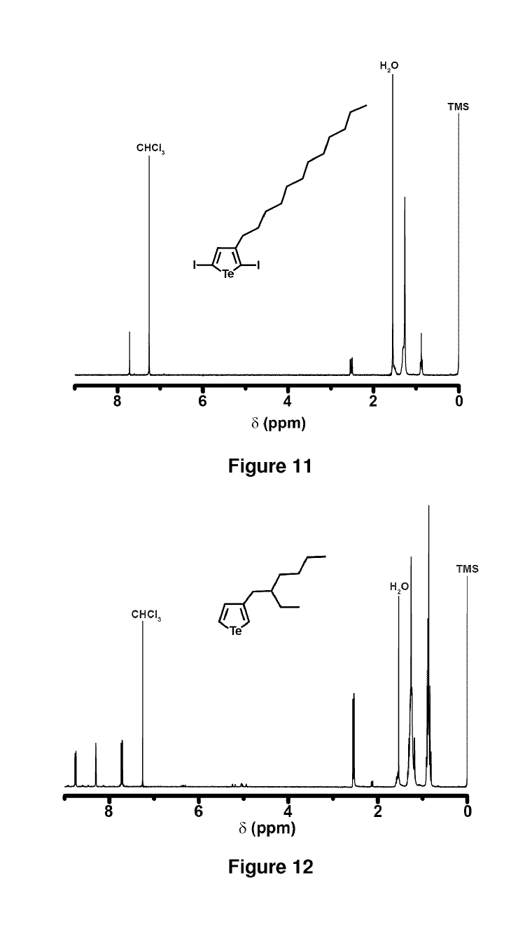

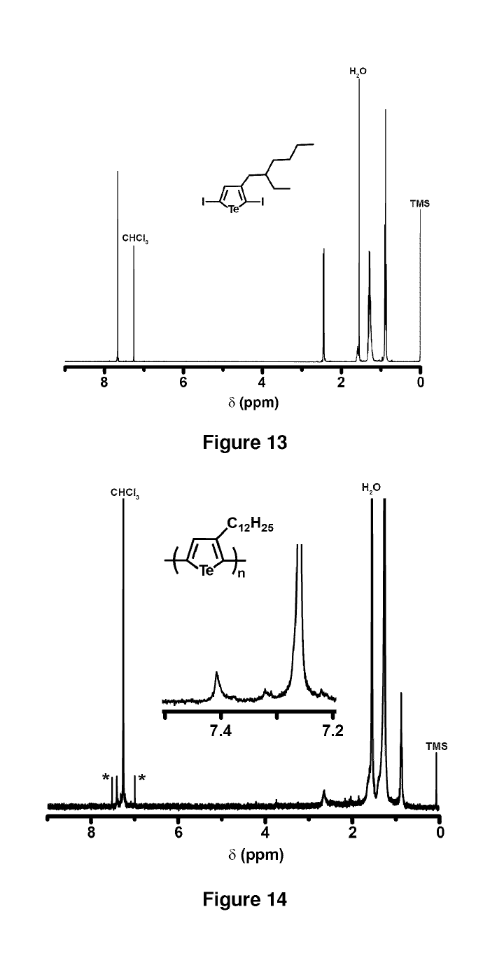

Another advantage of the polythiophenes (and polyselenophenes) is their ability to be synthesized under controlled chain-growth polymerization methods.¹⁵ This has led to the formation of narrow polydispersity homopolymers with relatively high molecular weight as well as distinct block-type¹⁶ and gradient-type¹⁷ copolymers. 3-alkyltellurophene compounds were iodinated in the 2- and 5-positions by treatment with sec-butyllithium followed by electrophilic quenching with iodine to afford 2,5-diiodo-3-alkyltellurophenes (6a-c; ¹H NMR spectra are shown in FIGS. 9, 11 and 13, respectively) for testing their ability to polymerize using a Kumada catalyst transfer polymerization. It was found possible to prepare polymers by activation with an isopropylmagnesium chloride lithium chloride complex, followed by addition of [1,3-bis(diphenylphosphino)propane]nickel(II) chloride catalyst. See the scheme shown in FIG. 4. Polymerization reactions were conducted in methyl THF at 80° C. to maintain the solubility of the growing chain and afford a high molecular weight polymer. Typical reaction times were 24 to 48 hours. After this time, the reaction mixtures were added to methanol to precipitate the polymer products which were then collected and purified by soxhlet extraction with various solvents, depending on the side-chain substituent, as described in greater detail in the Examples. For example, P3HTe was washed successively with methanol, hexanes, and chloroform before collecting the remaining insoluble material (the desired product). Poly(3-(2′-ethyl)hexyltellurophene) (P3EHTe) was much more soluble than P3HTe and was washed with methanol and ethyl acetate before being extracted in hexanes.

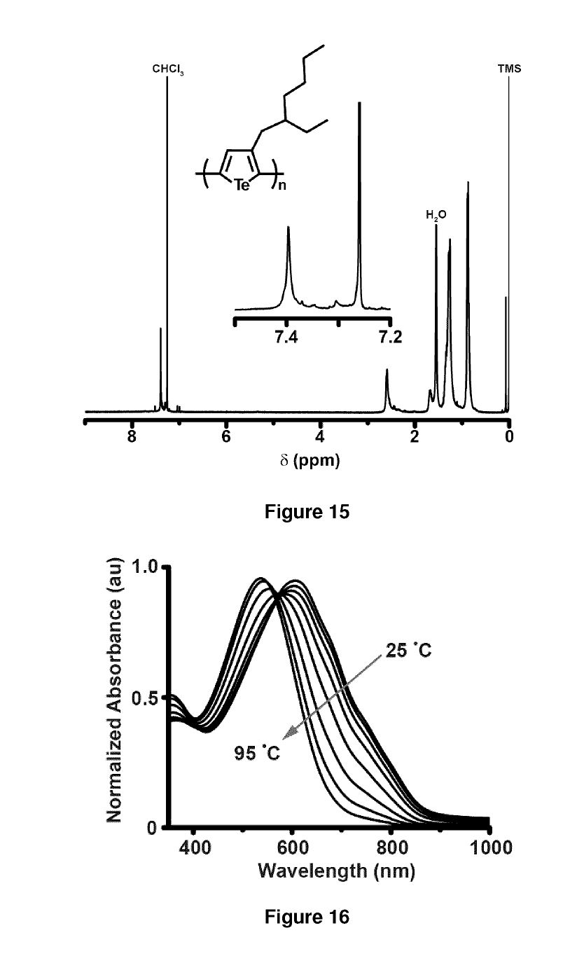

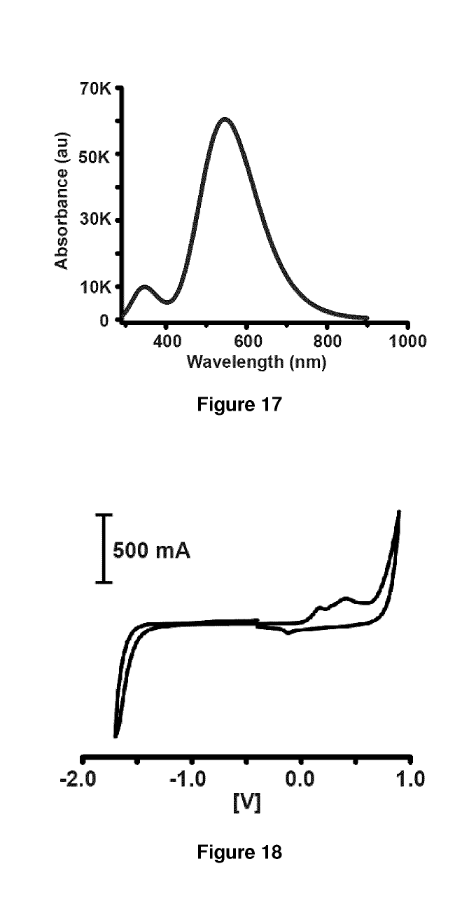

NMR was used to further characterize the polymers and determine if regioregular materials had been prepared. Regioregularity is significant in solid-state organization and charge transport properties. Poly(3-dodecyltellurophene) (P3DDTe; FIG. 14) and P3EHTe have ¹H NMR resonances at 7.40 ppm, which were assigned as the aromatic tellurophene proton. This is downfield from the aromatic resonances of poly(3-hexylthiophene) and poly(3-hexylselenophene) (P3HS), which are at 6.98 and 7.12 ppm, respectively. This is consistent with the trend that a heavier group-16 atom leads to a down-field shift in the aromatic resonance. Integration of the two methylene signals confirms that the P3EHTe obtained was 93% regioregular (FIGS. 5 and 15). P3DDTe did not exhibit a second (regiorandom) methylene peak in the proton spectra, which is indicative of a high degree of regioregularity. The signal was too weak to obtain an exact value. Due to solubility limitations, the ¹H NMR of P3HTe was not obtained.

Polymer chain length approximation by gel permeation chromatography relative to polystyrene standards (conducted in 1,2,4-tricholobenzene at 140° C.) was conducted to confirm the polymeric nature of the materials. These data show that P3HTe and P3DDTe have similar A4, values, 9.9 and 11.3 kDa, respectively, while P3EHTe was lower (5.4 kDa). The ethylhexyl side chains may hinder the nickel-catalyzed chain-growth due to steric effects, which offers an explanation of this trend.¹⁸ Based on the monomer:catalyst ratio a degree of polymerization of 100 was expected, leading to an Mₙ of 26-35 kDa for all of the exemplary polymers. Shorter than expected chains for all three polymers was likely due to chain termination before complete monomer consumption. This may be due to either solubility limitations or a weaker association of the Ni catalyst with the tellurophene chain. Given the lack of previously reported polytellurophenes, however, these molecular weights are reasonably high, and confirm that polymeric materials were prepared.

To better understand the properties of polytellurophenes, optical studies on all three polymer samples were performed in chlorobenzene. P3HTe and P3DDTe have maximum absorption peaks (558 nm and 545 nm, respectively) that occur at a notably longer wavelength than P3HT (455 nm) or P3HS (500 nm), which is consistent with theory that predicts that polytellurphenes will have a more narrow HOMO-LUMO gap than thiophenes and selenophenes. For P3EHTe, a blue-shift in absorption maximum (to 512 nm) was observed relative to P3HTe and P3DDTe, and could be due to backbone twisting that results from the bulky ethylhexyl side chain.¹⁹ Although P3HTe and P3DDTe have similar maximum absorption peaks, a well-defined shoulder was observed in the long wavelength region of the P3HTe absorbance spectrum. This may be attributed to the presence of aggregated chains that arise from the limited solubility of this polymer, consistent with NMR studies. Upon heating to 95° C. in 1,2,4-trichlorobenzene, P3HTe was fully dissolved and the shoulder no longer present. The molar absorptivities of the three polymers were obtained in chlorobenzene. P3HTe, P3DDTe, and P3EHTe have molar absorptivities of 3900, 5100, and 6400 M⁻¹ cm⁻¹ (calculated per repeat unit), respectively, revealing that all three polymers are strong light absorbers.

All three polymers were found to have a second, weaker, high energy absorption band in their solution absorption spectra. Consistent with this observation, time dependent density functional theory calculations predict a high-energy transition with an oscillator strength of 0.14 compared to 1.49 for the lower energy transition (See FIG. 6). The high-energy transition is a HOMO-1 to LUMO+1 transition while the lower energy transition is HOMO to LUMO. This high energy band is also predicted for P3HT and P3HS, but occurs at a lower wavelength and is therefore not often observed in the wavelength range that is typically reported for these polymers in solution.

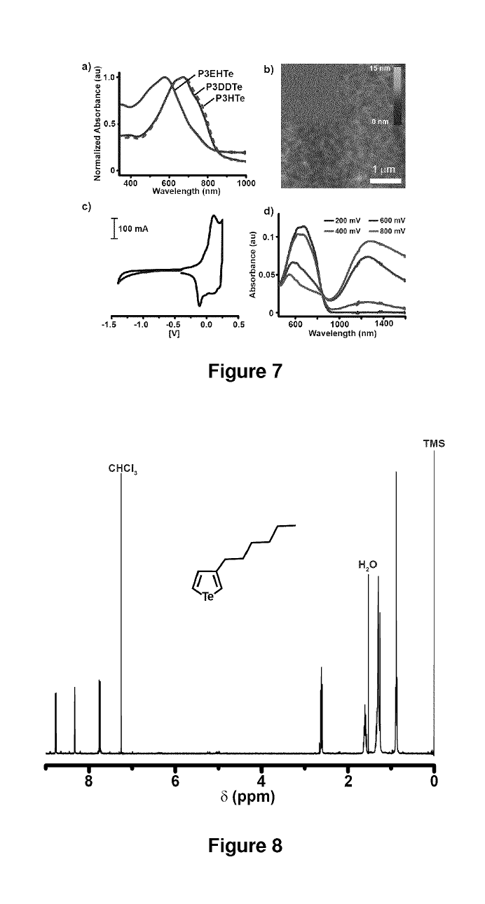

Solid-state properties of the exemplary polytellurophenes were also examined. Films were prepared by spin-casting solutions of polymers from hot chlorobenzene followed by annealing (100° C., 1 h), and then optical properties of the films were measured. P3HTe and P3DDTe have structured solid-state absorption spectra with long wavelength shoulders that are indicative of interchain π-stacking (FIG. 7(a)). This further supports the conclusion that these polymers are regioregular as only regioregular polyheterocycles have these characteristic vibronic peaks in their solid-state spectra. For P3EHTe, the long wavelength shoulder is not as well pronounced, which could be due to the shorter chain-length of the polymer or back-bone twisting as described earlier. The optical HOMO-LUMO gaps of P3HTe and P3DDTe, determined by onset of absorption, are 1.44 eV while P3EHTe has a 1.57 eV optical HOMO-LUMO gap. To probe the samples, further atomic force microscopy images were obtained. This showed that P3EHTe forms nanofibrils as a thin film (FIG. 7(b)). It thus appears that all three polymers are organized, at least to some degree, in the solid state.

Electrochemical properties of a P3HTe film spin coated onto an ITO working electrode were also examined. A reversible oxidation with an onset at 0.02 V was observed, followed by a second oxidation with an onset at 0.25 V (FIG. 7(c)). During reductive scanning, a peak with an onset around −1.35 V was also observed, indicating an electrochemical HOMO-LUMO gap of 1.37 eV, which is significantly narrower than polyselenophene.^{3(a)} The observed reversibility of the oxidative wave prompted us to conduct spectroelectrchemistry experiments on a film of P3HTe.

Stable oxidative (p-type) doping is a hallmark of robust and stable conjugated polymer materials. To further characterize properties and test whether the exemplary polymers can be doped, spectroelectrochemical properties of the chemically synthesized P3HTe film were examined. A well-defined absorption in the near infrared (IR) region of the spectrum appears upon oxidation and increases with potential, while a concurrent reduction of the absorbance in the visible region occurs (FIG. 7(d)). This IR absorption is characteristic of the formation of a polaron. Changes observed in the spectra are reversible up to potentials of 0.40 V, which demonstrates that P3HTe is stable towards electrochemical doping at this potential. At higher potentials, even larger changes in the near IR region were observed and the spectrum of the oxidized product remained stable for several successive scans. Overall, these data are indicative of stable oxidative doping.

### EXAMPLES

### Reagents and Materials

All reagents were used as received unless otherwise noted. N,O-Dimethylhydroxylamine hydrochloride, chloroacetyl chloride, para-toluenesulfonic acid monohydrate, hexylmagnesium bromide (2.0M in diethyl ether), dodecylmagnesium bromide (1.0M in THF), (2-ethylhexyl)magnesium bromide (1.0M in diethyl ether), dichloro[1,3-bis(diphenylphosphino)propane]nickel, ethynylmagnesium bromide (0.5M in THF), isopropylmagnesium chloride lithium chloride complex (1.3M in THF), N,N,N′,N′-tetramethylethylenediamine, sec-BuLi (1.4 M in cyclohexane), iodine, and tellurium were purchased from Sigma-Aldrich. Potassium hydroxide, sodium chloride, sodium thiosulfate, sodium bicarbonate, ammonium chloride, and magnesium sulfate were purchased from Fisher Scientific. Sodium borohydride was purchased from Acros Organics. 2-Chloro-N-methoxy-N-methylacetamide (1) was synthesized according to literature procedures.²⁰

### Instrumentation

Absorption spectra were recorded using a Varian Cary 5000 spectrometer. Solution measurements were made in chlorobenzene at ˜0.05 mg/mL. NMR spectra were recorded on a Varian Mercury 400 spectrometer (400 MHz). Masses were determined on a Waters GCT Premier ToF mass spectrometer (EI). Polymer molecular weights were determined in 1,2,4-trichlorobenzene at 140° C. using a Varian PL220 GPC that was referenced to narrow weight distribution polystyrene standards. AFM images were obtained with a Veeco Dimension 3000 microscope. Electrochemistry was performed with a BASi Epsilon potentiostat.

### Monomer Synthesis

### 1-Chloro-2-octanone (2a)

A solution of 2-chloro-N-methoxy-N-methylacetamide (10.00 g, 72.7 mmol) in 300 mL of dry THF at 0° C. was treated with 46 mL of hexylmagnesium bromide (2.0 M in diethyl ether). The mixture was allowed to slowly warm to room temperature and stir for 3 hours before being quenched with a 5% HCl solution. The reaction mixture was diluted with diethyl ether (300 mL) and the organic layer was washed once with saturated sodium bicarbonate (200 mL) and two times with brine (200 mL each). The organic layer was dried over MgSO₄ and concentrated under vacuum to give 10.86 g (92%) of the title compound, a yellow liquid that required no further purification. ¹H NMR (CDCl₃, 400 MHz): δ 4.07 (s, 2H), 2.58 (t, J=7.4 Hz, 2H), 1.61 (m, 2H), 1.28 (m, 6H), 0.88 (t, J=6.9 Hz, 3H). ¹³C NMR (CDCl₃, 100 MHz): δ 203.0, 48.3, 39.9, 31.6, 28.9, 23.7, 22.6, 14.1. HRMS-EI: calc. 163.0890, found 163.0892, Δ=1.2 ppm.

### 1-chloro-2-tetradecanone (2b)

Quantitative yield. ¹H NMR (CDCl₃, 400 MHz): δ 4.07 (s, 2H), 2.58 (t, J=7.4 Hz, 2H), 1.62 (m, 2H), 1.26 (m, 18H), 0.88 (t, J=6.9 Hz, 3H). ¹³C NMR (CDCl₃, 100 MHz): δ 190.1, 39.7, 31.9, 29.6, 29.4, 29.3, 23.6, 22.7, 14.1. HRMS-DART: M+[NH₄⁺] calc. 264.2094, found 264.2095, Δ=0.3 ppm.

### 1-chloro-4-ethyl-2-octanone (2c)

Quantitative yield. ¹H NMR (CDCl₃, 400 MHz): δ 4.07 (s, 2H), 2.50 (dd, J₁=6.7 Hz, J₁=0.7 Hz, 2H), 1.91 (m, 1H), 1.26 (m, 8H), 0.86 (t, J=7.4 Hz, 6H). ¹³C NMR (CDCl₃, 100 MHz): δ 202.9, 48.8, 44.3, 35.4, 33.3, 29.0, 26.5, 23.1, 14.2, 11.0. HRMS-DART: M+[NH₄⁺] calc. 208.1468, found 208.1469, Δ=0.2 ppm.

### 3-(chloromethyl)-1-nonyn-3-ol (3a)

A solution of 205 mL ethynylmagnesium bromide (0.5M in THF) at 0° C. was treated with a solution of 1-chloro-2-octanone (9.83 g, 60.4 mmol) in 30 mL of dry THF. The combined solution was stirred at 0° C. for 22 hours before being diluted with hexanes (300 mL) and quenched with saturated ammonium chloride. The organic layer was washed three times with brine (200 mL), dried over MgSO₄, and concentrated under vacuum to give 10.70 g (94%) of the title compound, a dark orange oil that was used without further purification. ¹H NMR (CDCl₃, 400 MHz): δ 3.70 (d, J=11 Hz, 1H), 3.60 (d, J=11 Hz, 1H), 2.55 (s, 1H), 2.51 (s, 1H), 1.74 (m, 2H), 1.57 (m, 2H), 1.31 (m, 6H), 0.89 (t, J=6.9 Hz, 3H). ¹³C NMR (CDCl₃, 100 MHz): δ 83.8, 73.8, 70.7, 53.14, 39.3, 31.8, 29.4, 24.2, 22.7, 14.2.

### 3-chloromethyl-1-pentadecyn-3-ol (3b)

98% yield. ¹H NMR (CDCl₃, 400 MHz): δ 3.70 (d, J=11 Hz, 1H), 3.60 (d, J=11 Hz, 1H), 2.58 (s, 1H), 2.51 (s, 1H), 1.74 (m, 2H), 1.57 (m, 2H), 1.26 (m, 18H), 0.88 (t, J=6.9 Hz, 3H). ¹³C NMR (CDCl₃, 100 MHz): δ 83.8, 73.8, 70.7, 53.2, 39.3, 32.1, 29.8,^{a} 29.7,^{b} 29.6, 29.5, 24.3, 22.8, 14.3. (a) 3 peaks at this resonance, (b) 2 peaks at this resonance. HRMS-DART: M+[NH₄⁺] calc. 290.2251, found 290.2262, Δ=3.7 ppm.

### 3-chloromethyl-5-ethyl-1-nonyn-3-ol (3c)

84% yield. ¹H NMR (CDCl₃, 400 MHz): δ 3.70 (d, J=10.9 Hz, 1H), 3.59 (d, J=10.9 Hz, 1H), 2.54 (s, 1H), 2.52 (s, 1H), 1.67 (m, 5H), 1.26 (m, 6H), 0.87 (t, J=3.7 Hz, 6H). ¹³C NMR (CDCl₃, 100 MHz): δ 84.1, 74.0, 70.9, 54.1, 42.6, 39.4, 33.1, 28.7, 26.9, 23.2, 14.3, 10.8.

### 3-Hexyltellurophene (5a)

Tellurium (12.25 g, 96 mmol) in degassed ethanol (350 mL) was treated with NaBH₄ (7.26 g, 186.6 mmol) under nitrogen in a 1 L 3-neck round bottom flask fitted with a condenser. The reaction was heated to reflux for 2.5 hours while an additional 7.26 g of NaBH₄ was added in 4 portions every 30 minutes. After this, the solution was cooled to 0° C. and a degassed solution of 3-(chloromethyl)-1-nonyn-3-ol (11.3 g, 60 mmol) in ethanol (20 mL) was added. The reaction was maintained at 0° C. for one hour before being warmed to room temperature. Next, a solution of potassium hydroxide (5.38 g, 96 mmol) in ethanol/water (50 mL/1.2 mL) was added and the reaction was heated to reflux for 2 hours. The reaction was quenched by stirring vigorously while exposed to laboratory air, and filtered through Celite. The solution was diluted with dichloromethane (400 mL) and washed three times with brine (300 mL), dried over MgSO₄, and concentrated under vacuum to give a dark orange oil. The oil was then redissolved in 60 mL of hexanes in a 250 mL round bottom flask fitted with a condenser. This solution was treated para-toluenesulfonic acid monohydrate (666 mg, 3.5 mmol) and the reaction was heated to reflux for 1 hour. The reaction was allowed to cool to room temperature before being diluted with hexanes (200 mL) and quenched with saturated sodium bicarbonate (50 mL). The hexanes layer was washed three times with brine (150 mL), dried over MgSO₄, and concentrated under vacuum to give 10.36 g of a dark orange oil that contained the desired product. Purification by column chromatography with hexanes afforded 5.80 g (37%) of the title compound, an orange oil. ¹H NMR (CDCl₃, 400 MHz): δ 8.78 (dd, J₁=1.88 Hz, J₂=6.6 Hz, 1H), 8.33 (s, 1H), 7.76 (dd, J₁=1.48 Hz, J₂=6.6 Hz, 1H), 2.62 (t, J=7.7 Hz, 2H), 1.61 (m, 2H), 1.30 (m, 6H), 0.88 (t, J=6.9 Hz, 3H). ¹³C NMR (CDCl₃, 100 MHz): δ 151.1, 140.2, 124.2, 117.9, 35.0, 31.9, 30.4, 29.1, 22.8, 14.3. HRMS-EI: calc. 266.0314, found 266.0319, Δ=1.9 ppm.

### 3-dodecyltellurophene (5b)

51% yield. ¹H NMR (CDCl₃, 400 MHz): δ 8.78 (dd, J₁=1.9 Hz, J₂=6.6 Hz, 1H), 8.33 (s, 1H), 7.76 (dd, J₁=1.5 Hz, J₂=6.6 Hz, 1H), 2.61 (t, J=7.7 Hz, 2H), 1.61 (m, 2H), 1.26 (b, 18H), 0.88 (t, J=6.7 Hz, 3H). ¹³C NMR (CDCl₃, 100 MHz): δ 140.2, 124.2, 117.9, 35.0, 32.1, 30.5, 29.8,^{a} 29.6, 29.5,^{b} 22.9, 14.3. (a) 4 peaks at this resonance, (b) 2 peaks at this resonance. HRMS-DART: calc. 351.1332, found 351.1323, Δ=2.4 ppm.

### 3-(2-ethylhexyl)tellurophene (5c)

30% yield. ¹H NMR (CDCl₃, 400 MHz): δ 8.76 (dd, J₁=1.9 Hz, J₂=6.6 Hz, 1H), 8.30 (s, 1H), 7.73 (dd, J₁=1.5 Hz, J₂=6.6 Hz, 1H), 2.55 (d, J=6.9 Hz, 2H), 1.59 (m, 1H), 1.26 (m, 8H), 0.86 (t, J=2.7 Hz, 6H). ¹³C NMR (CDCl₃, 100 MHz): δ 152.0, 145.9, 140.6, 123.9, 40.3, 39.2, 32.6, 29.0, 25.8, 23.2, 14.3, 11.0. HRMS-DART: calc. 295.0706, found 295.0715, Δ=3.2 ppm. 2,5-diiodo-3-hexyltellurophene (6a): Adapted from Sweat and Stephens.²¹ A solution of 3-hexyltellurophene (3 g, 11.4 mmol) and N,N,N′,N′-Tetramethylethylenediamine (3.6 ml, 23.9 mmol) in 35 mL of dry hexanes in a 100 mL Schlenk flask with a nitrogen atmosphere was treated dropwise with sec-BuLi (17.2 mL, 1.4 M in cyclohexane) at room temperature. The mixture was heated to 63° C. under nitrogen for 45 min. The flask was cooled to 0° C. and a solution of iodine (7.23 g, 28.5 mmol) in 55 mL of dry ether was added using a cannula. The reaction was allowed to stir at room temperature for 24 hours before being slowly quenched with water. The mixture was diluted with hexanes (100 mL) and the organic layer was washed one time with 10% sodium thiosulfate (100 mL) and three times with brine (100 mL), dried over MgSO₄, and concentrated under vacuum to give 2.80 g of a viscous brown oil. Purification by column chromatography with hexanes afforded 453 mg (8%) of yellow oil. ¹H NMR (CDCl₃, 400 MHz): δ 7.72 (s, 1H), 2.52 (t, J=7.8 Hz, 2H), 1.52 (m, 2H), 1.31 (b, 6H), 0.89 (t, J=6.9 Hz, 3H). ¹³C NMR (CDCl₃, 100 MHz): δ 149.2, 102.7, 70.8, 69.8, 36.3, 32.1, 30.3, 29.3, 23.1, 14.6. HRMS-EI: calc. 517.8247, found 517.8242, Δ=1.0 ppm. 2,5-diiodo-3-dodecyltellurophene (6b): 9% yield. ¹H NMR (CDCl₃, 400 MHz): δ 7.72 (s, 1H), 2.52 (t, J=7.7 Hz, 2H), 1.26 (b, 20H), 0.89 (t, J=6.7 Hz, 3H). ¹³C NMR (CDCl₃, 100 MHz): δ 157.5, 148.9, 70.5, 68.4, 36.0, 32.1, 30.0, 29.8,^{a} 29.7, 29.6, 29.5, 29.3, 22.9, 14.3. (a) 2 peaks at this resonance. HRMS-DART: calc. 602.9264, found 602.9251, Δ=2.2 ppm. 2,5-diiodo-3-(2-ethylhexyl)tellurophene (6c): 16% yield. ¹H NMR (CDCl₃, 400 MHz): δ 7.67 (s, 1H), 2.45 (d, J=7.3 Hz, 2H), 1.59 (m, 1H), 1.30 (m, 8H), 0.88 (t, J=7.1 Hz, 6H). ¹³C NMR (CDCl₃, 100 MHz): δ 156.8, 149.3, 71.3, 69.2, 40.4, 40.1, 32.5, 28.9, 25.8, 23.2, 14.3, 11.1. HRMS-DART: calc. 546.8638, found 546.8637, Δ=0.2 ppm.

### Typical Polymerization

### Poly(3-hexyltellurophene)

A solution containing Isopropylmagnesium chloride lithium chloride complex (0.89 mL, 1.3 M in THF, 1.16 mmol) was added to a solution of 2,5-diiodo-3-hexyltellurophene (600 mg, 1.16 mmol) in dry methyl THF (9 mL) under a nitrogen atmosphere. The mixture was stirred for 30 minutes at room temperature, then transferred to a flask containing [1,3-bis(diphenylphosphino)propane]nickel(11) chloride (6.3 mg, 0.0116 mmol). The solution was heated to 80° C. for 24 hours then quenched by precipitation into methanol. The polymer was purified by soxhlet extraction with methanol, hexanes, and chloroform. The remaining insoluble material, the desired product, a purple solid, was collected (101 mg, 33%). λₘₐₓ=558 nm, Mₙ=9.9 kDa, M_{w}=21.8 kDa, PDI=2.2.

### Poly(3-dodecyltellurophene)

Prepared in an analogous manner as poly(3-hexyltellurophene). Purified by soxhlet extraction with methanol hexanes and dichloromethane. The product was collected by extraction in chloroform (143 mg, 62% yield). ¹H NMR (CDCl₃, 400 MHz): δ 7.41 (s, 1H), 2.65 (b, 2H), 1.25 (m, 20H), 0.88 (t, J=1.6 Hz, 3H). λₘₐₓ=545 nm, Mₙ=11.3 kDa, M_{w}=22.9 kDa, PDI=2.0.

### Poly(3-(2′-ethylhexyl)tellurophene)

Prepared in an analogous manner as poly(3-hexyltellurophene) with the exception that the polymerization allowed to react at 80° C. for 48 h. Purified by soxhlet extraction with methanol and ethyl acetate. The product was collected by extraction in chloroform (56 mg, 35% yield). ¹H NMR (CDCl₃, 400 MHz): δ 7.41 (s, 1H), 2.60/2.45 (d, J=1.7 Hz, 2H), 1.68 (b, 1H), 1.26 (m, 8H), 0.88 (t, J=1.5 Hz, 6H). λₘₐₓ=512 nm, Mₙ=5.4 kDa, M_{w}=10.0 kDa, PDI=1.9.

### Film Preparation

Glass substrates were prepared by washing with detergent and rinsing with distilled water followed by methanol. Indium tin oxide substrates were prepared by washing with detergent followed by sonication in distilled water, acetone, and methanol. Solutions of polymers in chlorobenzene (5 mg/mL) were heated with a heat gun until the color had changed to bright red, signifying that all polymer was dissolved. This solution was deposited onto a substrate by spin-casting (1000 RPM, 30 s). The films used for absorbance measurements were annealed at 150° C. for one hour in a nitrogen atmosphere.

### Electrochemical Measurements

All electrochemical measurements were performed at room temperature with a BASi Epsilon electrochemical workstation using anhydrous acetonitrile (or anhydrous DCM for polymerization spectroelectrochemistry) containing 0.5 M supporting electrolyte (Bu₄NPF₆). A typical three-electrode setup was used including a platinum or ITO working electrode, Ag wire reference electrode and a platinum wire auxiliary electrode and ferrocene was used as an internal standard in all cases (ferrocene vs. Ag=0.40 V).

Additional embodiments of the invention are described in light of the Examples.

An “alkyl” group is the radical obtained when one hydrogen atom is removed from a hydrocarbon. An alkyl group can have from 1 to 100 carbon atoms, or 1 to 50, 10 to 25, 1 to 20, 1 to 12, 1 to 6, or 1 to 4 carbon atoms. The term includes the normal i.e., linear alkyl (n-alkyl), secondary and tertiary alkyl, so can be straight-chain or branched. For any use of the term “alkyl”, unless clearly indicated otherwise, it is intended to embrace all variations of alkyl groups disclosed herein, as measured by the number of carbon atoms, the same as if each and every alkyl group were explicitly and individually listed for each usage of the term. When an alkyl residue having a specific number of carbons is named, all geometric isomers having that number of carbons are included, so, for example, “butyl” includes n-butyl, sec-butyl, iso-butyl and t-butyl. Examples of alkyl groups are methyl, ethyl, n-propyl, isopropyl, n-butyl, isobutyl, s-butyl, t-butyl, pentyl, isopentyl, hexyl, particularly —CH₂CH₂CH₂CH₂CH₂CH₃, isohexyl, dodecyl, particularly —(CH₂)₁₁CH₃, icosyl, —CH₂CH(C₂H₅)(CH₂CH₂CH₂)CH₃, etc.

The term alkyl group includes “cycloalkyl” which indicates a saturated cycloalkane radical having 3 to 20 carbon atoms, or 3 to 10 carbon atoms, in particular 3 to 8 carbon atoms, such as 3 to 6 carbon atoms, including fused bicyclic rings, e.g. cyclopropyl, cyclobutyl, cyclopentyl, cyclohexyl, or cycloheptyl.

A “heteroalkyl” group is an alkyl radical as described above in which one or more carbon atoms, —CH groups, —CH₂— groups or —CH₃ groups is replaced by a heteroatom. Heteroatoms are O, S, N, Se, P, B, Cl, F, I, Br, Si, Ge, Te and Sn. Examples of heteroalkyl groups are —CH₂OCH₂CH₃ or —OCH₂CH₂CH₃ in which a CH₂ group of —CH₂CH₂CH₂CH₃ is replaced by an oxygen atom; —CH₂NHCH₂CH₃ (or —CH₂N(CH₃)₂ in which a CH group of —CH₂CH₂CH₂CH₃ (or —CH₂CH(CH₃)₂) is replaced by a nitrogen atom; —CH₂CHFCH₂CH₃ in which a CH₃ group of —CH₂CH(CH₃)CH₂CH₃ is replace by fluorine. The number of permitted substitutions is 1, 2, 3, 4, 5, 6, 7, 8, 9, 10, 11, 12, 13, 14, 15, 16, 17, 18, 19 or 20, the number being less than the number of carbon atoms of the alkyl radical from which the heteroalkyl group is derived.

It is noted here, that when discussing radical portions of a molecule, such as “CR₃” or “F” or “CH₂”, etc. the connecting bond(s) may be omitted in various contexts for the sake of convenience, as for example when the location of a bond or bonds is unambiguous, and the skilled person understands this.

A “heterocycloalkyl” group is a cycloalkane radical as described above in which one or more carbon atoms, —CH groups, —CH₂— groups or —CH₃ groups is replaced by a heteroatom. The number of substitutions is 1, 2, 3, 4, 5, or 6. Examples of molecules from which heterocycloalkyl radicals are derived are [1,3]dioxole, oxetane, [1,3]dioxolane, [1,3]dioxane, tetrahydrothiopyran, tetrahydrothiopyran-1,1-dioxide, tetrahydrothiopyran-1-oxide, N-methylpiperidine, piperidine, tetrahydrothiophene, [1,3]-dithiane, thietane, [1,3]-dithiane-1,3-dioxide, or thietane-1-oxide. Fused bicyclic rings with 1 to 4 heteroatoms, wherein at least one ring includes a heteroatom are included, for example, isoindolyl.

An “aryl” group indicates a radical of an aromatic carbocyclic ring(s) having 6 to 20 carbon atoms, such as 6 to14 carbon atoms, 6 to 10 carbon atoms, or 6-membered rings, and an aromatic ring or rings may be fused with at least one other aromatic ring, such as phenyl, naphthyl, indenyl and indanyl.

The term “heteroaryl” indicates a radical of one or more aromatic rings having 1 to 6 heteroatoms (O, S, N, Se, Si, Te) and 1 to 20 carbon atoms, such as 1 to 6 heteroatoms and 1 to 10 carbon atoms, or 1 to 5 heteroatoms and 1 to 6 carbon atoms, or 1 to 5 heteroatoms and 1 to 3 carbon atoms e.g., 5- or 6-membered rings with 1 to 4 heteroatoms selected from O, S and N. Included are fused bicyclic rings with 1 to 4 heteroatoms, in which at least one ring is aromatic, e.g. pyridyl, quinolyl, isoquinolyl, indolyl, tetrazolyl, thiazolyl, imidazolyl, pyrazolyl, oxazolyl, isoxazolyl, thienyl, pyrazinyl, isothiazolyl, benzimidazolyl and benzofuranyl.

Alkyl, heteroalkyl, cycloalkyl, heterocycloalkyl, aryl and heteroaryl groups can be optionally substituted with one or more of the groups described above and/or one or more of nitro, carboxyl, formyl, —C(O)—R¹ in which the R¹ group of —C(O)—R¹ can be alkyl, cycloalkyl, heteroalkyl, heterocycloalkyl, aryl or heteroaryl. These latter substitutions can be seen as replacement of a carbon-bound hydrogen atom of the group from which the substituted radical is derived.

In embodiments, the invention provides a compound having formula (5):

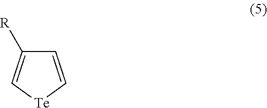

in which R is a monovalent organic group.

The substituent, R, covalently linked to the telluropene ring can be:
alkyl, optionally substituted with one or more of cycloalkyl, heteroalkyl, heterocycloalkyl, aryl, heteroaryl, nitro, carboxyl, formyl, and —C(O)—R¹ in which R¹ is alkyl, cycloalkyl, heteroalkyl, heterocycloalkyl, aryl and heteroaryl;
cycloalkyl, optionally substituted with one or more of alkyl, cycloalkyl, heteroalkyl, heterocycloalkyl, aryl, heteroaryl, nitro, carboxyl, formyl, and —C(O)—R¹ in which R¹ is alkyl, cycloalkyl, heteroalkyl, heterocycloalkyl, aryl and heteroaryl;
heteroalkyl optionally substituted with one or more of cycloalkyl, heterocycloalkyl, aryl, heteroaryl, nitro, carboxyl, formyl, and —C(O)—R¹ in which R¹ is alkyl, cycloalkyl, heteroalkyl, heterocycloalkyl, aryl and heteroaryl;
heterocycloalkyl optionally substituted with one or more of alkyl, heteroalkyl, aryl, heteroaryl, nitro, carboxyl, formyl, and —C(O)—R¹ in which R¹ is alkyl, cycloalkyl, heteroalkyl, heterocycloalkyl, aryl and heteroaryl;
aryl optionally substituted with one or more of alkyl, cycloalkyl, heteroalkyl, heterocycloalkyl, aryl, heteroaryl, nitro, carboxyl, formyl, and —C(O)—R¹ in which R¹ is alkyl, cycloalkyl, heteroalkyl, heterocycloalkyl, aryl and heteroaryl; and
heteroaryl optionally substituted with one or more of alkyl, cycloalkyl, heteroalkyl, heterocycloalkyl, aryl, heteroaryl, nitro, carboxyl, formyl, and —C(O)—R¹ in which R¹ is alkyl, cycloalkyl, heteroalkyl, heterocycloalkyl, aryl and heteroaryl.

In the above context, the one or more substitutions are made independently of each other and multiple substitutions of the same substituent are included. For example, a substituted aryl group might have multiple nitro substituents in addition to any substitutions with other groups that are permitted.

In cases where a compound is capable of forming a salt e.g., contains —NH₂, such salts are included within the family of described compounds.

R-groups of the Examples fall into the category of groups in which R is C1-C20 alkyl.

In other embodiments, the invention provides a compound having formula (4):

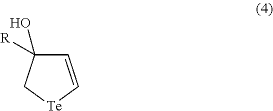

As illustrated in the Examples, such a compound is useful, for example, in the synthesis of a compound of formula (5) in which the R-group shown in formula (5) and (4) correspond to each other.

In another embodiment, a compound of the invention comprises two or more tellurophene-2,5-diyl groups covalently linked to each other at one or the other of the 2- and 5-positions of each tellurophene ring, wherein each of the tellurophene rings is substituted at the 3- or 4-position thereof.

Put another way, the invention includes a compound that includes structural units of formula (A):

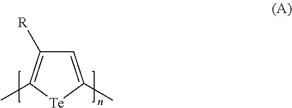

where n is an integer greater than 1.

According to certain embodiments, each R of compound A is, independently of the other, as described above for a compound of formula (5). In particular embodiments, such as those of the Examples, the R-group is the same for all n structural units i.e., monomeric tellurophene units of the compound are the same as each other. A homopolymer is a polymer in which the units are the same as each other.

Oligomers and polymers made up of tellurophene monomers linked together at the 2- and 5-positions of the tellurophene rings are fully conjugated.

As mentioned, the value of the integer n is greater than or equal to 2. The value of n in various embodiments is between 10 and 5,000, or between 10, and 4,000, or between 10 and 3,000, or between 10 and 2,000, or between 10 and 1,000, or between 10 and 500, or between 10 and 200, or between 20 and 180, or between 30 and 180, or between 20 and 150, or is about 10, about 20, about 30, about 40, about 50, about 60, about 70, about 80, about 90, about 100, about 110, about 120, about 130, about 140, about 150, about 160, about 170, about 180, about 190 or about 200.

In other embodiments, the value of n is greater than 10 and the compound has a regioregularity of at least 50%, or at least 55%, or at least 60%, or at least 65%, or at least 70%, or at least 75%, or at least 80%, or at least 85%, or at least 90%, or at least 91%, or at least 92%, or at least 93%, or at least 94%, or at least 95%, or at least 96%, or at least 97%, or at least 98% or at least 99%.

In embodiments, the value of n is greater than 10 and the compound is a polymer having a regioregularity between 50% and 100%, between 50% and 99%, between 70 and 99%, between 90 and 99%, between 50% and 95%, between 60% and 95%, between 50 and 93%, between 60% and 93%, between 65% and 100%, between 65% and 95%, between 65% and 93%, between 70% and 100%, between 70% and 95%, between 70% and 93%, between 75% and 100%, between 75% and 95%, between 75% and 93%, or between 80% and 95%.

In embodiments, the value of n is greater than 10 and the compound is a polymer having a regioregularity of about 50%, about 55%, about 60%, about 65%, about 70%, about 75%, about 80%, about 85%, about 90%, about 93%, about 94%, about 95%, about 96%, about 97%, about 98%, about 99% or about 100%.

In embodiments, the compound is a polymer, particularly a homopolymer, having a regioregularity of at least 50%, or at least 55%, or at least 60%, or at least 65%, or at least 70%, or at least 75%, or at least 80%, or at least 85%, or at least 90%, or at least 91%, or at least 92%, or at least 93%, or at least 94%, or at least 95%, or at least 96%, or at least 97%, or at least 98% or at least 99%.

In other embodiments, the compound is a polymer, particularly a homopolymer, having a regioregularity between 50% and 100%, between 50% and 99%, between 70 and 99%, between 90 and 99%, between 50% and 95%, between 60% and 95%, between 50 and 93%, between 60% and 93%, between 65% and 100%, between 65% and 95%, between 65% and 93%, between 70% and 100%, between 70% and 95%, between 70% and 93%, between 75% and 100%, between 75% and 95%, between 75% and 93%, or between 80% and 95%.

In other embodiments, the compound is a polymer, particularly a homopolymer, having a regioregularity of about 50%, about 55%, about 60%, about 65%, about 70%, about 75%, about 80%, about 85%, about 90%, about 93%, about 94%, about 95%, about 96%, about 97%, about 98%, about 99% or about 100%.

In embodiments, the compound is a polymer, particularly a homopolymer, having a number average molecular weight (Mₙ) that is at least 2,000, or at least 5,000, or at least 10,000, or at least 20,000 when measured by gel permeation chromatography relative to polystyrene standards. Suitably chosen polystyrene standards up to 1,000,000, likely between 20,000 and 100,000 can be used.

In embodiments, the invention includes a polymer in which Mₙ of the polymer is up to 1,000,000, or up to 500,000, or up to 400,000, or up to 300,000, or up to 200,000, or up to 150,000, or up to 120,000, or up to 100,000. Mₙ can be between 5,000 and 500,000, or between 5,000 and 400,000, or between 10,000 and 300,000, or between 15,000 and 200,000, or between 15,000 and 150,000, or between 20,000 and 100,000. Mₙ can be about 20,000, or about 30,000, or about 40,000, or about 50,000, or about 60,000, or about 70,000, or about 80,000, or about 90,000, or about 100,000.

Embodiments include a polymer in which Mₙ/M_{w} is between 1 and 3, or between 1 and 2.5, or between 1 and 2.0 or between 1 and 1.5, or in which Mₙ/M_{w} is about 1 or about 1.1 or about 1.2 or about 1.3 or about 1.4 or about 1.5 or about 1.6 or about 1.7 or about 1.8 or about 1.9 or about 2.0 or about 2.1 or about 2.2 or about 2.3 or about 2.4 or about 2.5.

In embodiments, the invention includes a film comprising a polymer, particularly a homopolymer. A film can have a thickness of between 1 and 10,000 nm, or between 10 and 5,000 nm, or between 20 and 500 nm, or between 40 and 400 nm, or between 40 and 300 nm, or a film can have a thickness of about 40 nm or about 50 nm, or about 60 nm, or about 70 nm, or about 80 nm, or about 90 nm, or about 100 nm, or about 110 nm, or about 120 nm, or about 130 nm, or about 140 nm, or about 150 nm, or about 160 nm, or about 170 nm, or about 180 nm, or about 190 nm, or about 200 nm, or about 210 nm, or about 220 nm, or about 230 nm, or about 240 nm, or about 250 nm, or about 260 nm, or about 270 nm, or about 280 nm, or about 290 nm, or about 300.

In embodiments, the invention includes a composite material comprising a polymer layer and a support disposed on at least one side of the polymer layer.

An embodiment is an optoelectronic device comprising the composite material. Such devices include a diode, a light-emitting diode, a transistor, a solar cell, a photodiode or a light-emitting transistor.

An electrode can be installed in contact with a film. The electrode can be part of a solar cell.

A semiconductor composite material can contain a polymer in combination with an electron acceptor material.

Embodiments include use of a compound of formula (B) in the preparation of other compounds, particularly oligomeric and polymeric compounds. The Examples describe synthesis of homopolymers.

In one embodiment, such preparation includes use of compound having formula (B):

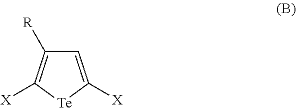

where R is as described above.
each X is, independently of the other X, F, Cl, Br, I, H, Li, Na, MgX¹, B(OR′)(OR″), B(OH)₂, or SnR′″₃,
X¹ is CI or Br,
R′ and R″ for B(OR′)OR″) may be the same or different as each other, and each can be any alkyl chain up to ten carbons or R′ and R″ can together bridge the oxygen atoms by a carbon chain up to ten carbons. The bridged chain may be substituted or unsubstituted with any hydrocarbon group, common examples being 1,3-propanediol ester, catechol ester, pinacol ester,
each R′″₃ is the same or different as the other and each is C1-C10 alkyl, and
if one X is H, then the other X is not H.

Compound (B) is activated as through the production of an organometallic intermediate followed by coupling of the activated compound using a coordination catalyst.

In the Examples, monomer (B) is activated using an isopropylmagesium chloride lithium chloride complex, but many such activating agents are known, such as isopropylmagnesium chloride, hexylmagnesium bromide, tert-butyl magnesium bromide, methylmagnesium bromide, butylmagnesium bromide, or any alkylmagnesium halide (bromide or chloride).

In the Examples, the activated intermediate is combined with [1,3-bis(diphenylphosphino)propane]nickel(II) chloride, a coordination catalyst containing transition metal nickel. These activation and coupling steps are carried out in the Examples without isolating the activated monomer. Many coordination catalysts are known. Common catalysts include dichloro[1,3-bis(diphenylphosphino)propane]nickel, and dichloro[1,3-bis(diphenylphosphino)ethane]nickel, but any suitable Ni, Pd, Ir complex or nanoparticle can be used as a catalyst.

As the catalyzed coupling of activated sites proceeds, monomers become covalently linked to ends of a growing chain, often referred to as chain growth polymerization, and chain growth proceeds to form an oligomer or polymer. So, in one embodiment, the invention is a method for preparing a polymer, the method comprising:
(i) activating a monomer of formula (B) at the 2- and 5-positions of the tellurophene ring; and
(ii) polymerizing the activated monomer in the presence of a coordination catalyst.

Also described above, is the formation of a polymer by means of electrochemical polymerization of a compound having formula (5). It is thus possible to form a polymer as represented by formula (A) by exposing a compound of formula (5) to an electrochemical potential of from 0.1 to 3.0 V for a period of time sufficient to form the compound e.g., between 1 and 10,000 seconds. Other positive potentials can be used including about 0.1 V, about 0.2 V, about 0.4V, about 0.6V, about 0.8V, about 1V, about 1.2V, about 1.4V, about 1.6V, about 1.8V, about 2V, about 2.2V, about 2.4V, about 2.6V, about 2.8V or about 3.0 V. Once prepared, a polymer can be used, for example, in the preparation of a film by application to a substrate for incorporation into an optoelectronic device, such as a diode, a light-emitting diode, a transistor, a solar cell, a photodiode or a light-emitting transistor.

Application of the polymer to form a film typically includes taking a conjugated polymer described herein up in a solvent or solution in which it is soluble. In the case of the Example described herein, a polymer was dissolved in chlorobenzene and applied by spin-casting to a substrate. Other methods of polymer application, such as drop casting, doctor blading, ink jet printing, evaporation are known. Typically, the polymer film is then annealed by the application of heat. In the case of the Examples described herein, films were annealed at 150° C. for about an hour.

Polymers are applied to a substrate to obtain a desired thickness.

An embodiment of the invention is an article comprising a polymer film as described herein.

An article can be an electrode installed in contact with the film in the manufacture of a solar cell. An exemplary substrate in this case is a conductor layer such as indium tin oxide coated with PEDOT:PSS. A polymer solution can be applied directly to the conductor layer.

The polymer solution applied to a substrate can have admixed therewith an electron acceptor. An electron acceptor can be one or more of a fullerene, a fullerene derivative, a nanoparticle, nanocrystal, quantum dot, etc. Exemplary quantum dots include one or more of e.g., CdSe, CdTe, CdS, PbS, PbSe, CuInS₂, CuInSe₂, Cd₃As₂, Cd₃P₂.

Embodiments include methods of preparation of a compound of formula (5). In one aspect, such method includes the step of dehydrating a compound of formula (4) to form the compound of formula (5).

An embodiment of the invention is a method of preparing a compound of formula (2)

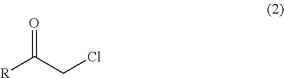

that includes coupling a compound of formula (1′)

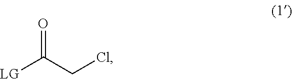

wherein LG is a leaving group, and an organometallic salt of the formula R⁻Z⁺. In the Examples, LG is —N(OH)R^{A} where R^{A} is an alkyl group that is methyl.

The invention includes a method of preparing a compound of formula (3)

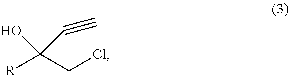

that includes coupling a compound of formula (2)

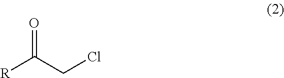

and an organometallic salt of the formula HC≡C⁻Z⁺.

The invention includes a method of preparing a compound of formula (4)

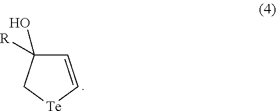

This is accomplished by:
(i) admixing a compound of formula (3)

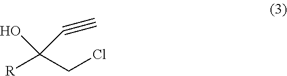

with a mixture of a tellurium salt and a reducing agent. In the Examples, the tellurium salt is Na₂Te.

The invention includes a method of preparing a compound of formula (4)

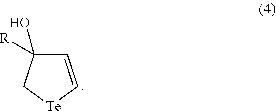

This can be accomplished by:
(i) coupling a compound of formula (1)

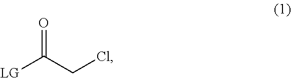

wherein LG is a leaving group, and an organometallic salt of the formula R⁻Z⁺ to form a compound of formula (2)

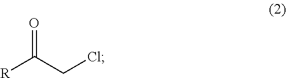

(ii) coupling the compound of formula (2) obtained in step (i) and an organometallic salt having of the formula HC≡C⁻Z⁺ to form a compound of formula (3)

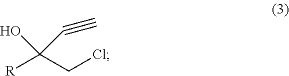

and
(iii) admixing the compound of formula (3) obtained in step (ii) with a mixture of a tellurium salt and a reducing agent.

This invention may also be said broadly to be composed of the parts, elements and features referred to or indicated herein, individually or collectively, in their various possible combinations. It is to be understood that those combinations and e.g., subranges are described as though each is explicitly described herein. For example, formula (A) defines a family of compounds, in which n is an integer greater than 1, and it is also said that n can be a number from 2 to 200. This is to be understood as though the full range of individual numbers 2, 3, 4, 5 . . . 200 had been written, and as though subranges of the numbers e.g., 2 to 24, 4 to 18, etc. had been written, and are included in combination with other such combinations, subcombinations, ranges, and subranges falling within those described herein.

The entire disclosures of all applications, patents and publications cited herein are hereby incorporated by reference.

### REFERENCES

(1) (a) McCullough, R. D. Adv. Mater. 1998, 10, 93. (b) Osaka, I.; McCullough, R. D. Acc. Chem. Res. 2008, 41, 1202. (c) Yokoyama, A.; Miyakoshi, R.; Yokozawa, T. Macromolecules 2004, 37, 1169.
(2) (a) Kaur, S.; Findlay, N. J.; Kanibolotsky, A. L.; Elmasly, S. E. T.; Skabara, P. J.; Berridge, R.; Wilson, C.; Coles, S. J. Polym. Chem. 2012, 3, 2277. (b) Ferrón, C. C.; Delgado, M. C. R.; Gidron, O.; Sharma, S.; Sheberla, D.; Sheynin, Y.; Bendikov, M.; Navarrete, J. T. L.; Hernandez, V. Chem. Commun. 2012, 48, 6732. (c) Bijleveld, J. C.; Karsten, B. P.; Mathijssen, S. G. J.; Wienk, M. M.; de Leeuw, D. M.; Janssen, R. A. J. J. Mater. Chem. 2011, 21, 1600.
(3) (a) Heeney, M.; Zhang, W.; Crouch, D. J.; Chabinyc, M. L.; Gordeyev, S.; Hamilton, R.; Higgins, S. J.; McCulloch, I.; Skabara, P. J.; Sparrowe, D.; Tierney, S. Chem. Commun. 2007, 5061. (b) Patra, A.; Wijsboom, Y. H.; Zade, S. S.; Li, M.; Sheynin, Y.; Leitus, G.; Bendikov, M. J. Am. Chem. Soc. 2008, 130, 6734. (c) Chen, Z.; Lemke, H.; Albert-Seifried, S.; Caironi, M.; Nielsen, M. M.; Heeney, M.; Zhang, W. M.; McCulloch, I.; Sirringhaus, H. Adv. Mater. 2010, 22, 2371. (d) Hollinger, J.; Jahnke, A. A.; Coombs, N.; Seferos, D. S. J. Am. Chem. Soc. 2010, 132, 8546. (e) Patra, A.; Bendikov, M. J. Mater. Chem. 2010, 20, 422. (f) Lee, W.-H.; Son, S. K.; Kim, K.; Lee, S. K.; Shin, W. S.; Moon, S.-J.; Kang, I.-N. Macromolecules 2012, 45, 1303. (g) Khim, D.; Lee, W.-H.; Baeg, K.-J.; Kim, D.-Y.; Kang, I.-N.; Noh, Y.-Y. J. Mater. Chem. 2012, 22, 12774. (h) Atak, S.; Içli-Özkut; Önal, A. M.; Cihaner, A. J. Polym. Sci., Part A: Polym. Chem. 2011, 49, 4398. (i) Li, L.; Hollinger, J.; Jahnke, A. A.; Petrov, S.; Seferos, D. S. Chem. Sci. 2011, 2, 2306. (j) Klein, M. F. G.; Pfaff, M.; Müller, E.; Czolk, J.; Reinhard, M.; Valouch, S.; Lemmer, U.; Colsmann, A.; Gerthsen, D. J. Polym. Sci., Part B: Polym. Phys. 2012, 50, 198. (k) Hollinger, J.; DiCarmine, P. M.; Karl, D.; Seferos, D. S. Macromolecules 2012, 45, 3772.
(4) (a) Inoue, S.; Jigami, T.; Nozoe, H.; Aso, Y.; Ogura, F.; Otsubo, T. Heterocycles 2000, 52, 159. (b) Saito, H.; Ukai, S.; Iwatsuki, S.; Itoh, T.; Kubo, M. Macromolecules 1995, 28, 8363. (c) Ng, S. C.; Ding, H.; Chan, H. S. O. Chem. Lett. 1999, 1325. (d) Jahnke, A. A.; Howe, G. W.; Seferos, D. S. Angew. Chem. Int. Ed. 2010, 49, 10140. (e) Jahnke, A. A.; Seferos, D. S. Macromolecular Rapid Communications 2011, 32, 943. (f) Lapkowski, M.; Motyka, R.; Suwinski, J.; Data, P. Macromol. Chem. and Phys. 2012, 213, 29.
(5) GaussView, Version 5, Dennington, R.; Keith, T.; Millam, J. Semichem Inc., Shawnee Mission Kans., 2009.
(6) M. J. Frisch, G. W. T., H. B. Schlegel, G. E. Scuseria, M. A. Robb, J. R. Cheeseman, G. Scalmani, V. Barone, B. Mennucci, G. A. Petersson, H. Nakatsuji, M. Caricato, X. Li, H. P. Hratchian, A. F. Izmaylov, J. Bloino, G. Zheng, J. L. Sonnenberg, M. Hada, M. Ehara, K. Toyota, R. Fukuda, J. Hasegawa, M. Ishida, T. Nakajima, Y. Honda, O. Kitao, H. Nakai, T. Vreven, J. A. Montgomery, Jr., J. E. Peralta, F. Ogliaro, M. Bearpark, J. J. Heyd, E. Brothers, K. N. Kudin, V. N. Staroverov, R. Kobayashi, J. Normand, K. Raghavachari, A. Rendell, J. C. Burant, S. S. Iyengar, J. Tomasi, M. Cossi, N. Rega, J. M. Millam, M. Klene, J. E. Knox, J. B. Cross, V. Bakken, C. Adamo, J. Jaramillo, R. Gomperts, R. E. Stratmann, O. Yazyev, A. J. Austin, R. Cammi, C. Pomelli, J. W. Ochterski, R. L. Martin, K. Morokuma, V. G. Zakrzewski, G. A. Voth, P. Salvador, J. J. Dannenberg, S. Dapprich, A. D. Daniels, Ö. Farkas, J. B. Foresman, J. V. Ortiz, J. Cioslowski, and D. J. Fox, Gaussian 09, Revision B.01, Gaussian, Inc.: Wallingford Conn., 2009.
(7) Becke, A. D., Density-functional thermochemistry. III. The role of exact exchange. J. Chem. Phys. 1993, 98, 5648-52.
(8) Stratmann, R. E.; Scuseria, G. E.; Frisch. M. J. J. Chem. Phys., 109 (1998) 8218-24.
(9) (a) Das, S.; Zade, S. S. Chem. Commun. 2010, 46, 1168. (b) Das, S.; Bedi, A.; Krishna, G. R.; Reddy, C. M.; Zade, S. S. Org. Biomol. Chem. 2011, 9, 6963. (c) Chattopadhyaya, M.; Sen, S.; Alam, M. M.; Chakrabarti, S.; J. Chem. Phys.: 2012; Vol. 136, p 094904.
(10) Zade, S. S.; Zamoshchik, N.; Bendikov, M. Chemistry—a European Journal 2009, 15, 8613.
(11) McCormick, T. M.; Jahnke, A. A.; Lough, A. J.; Seferos, D. S. J. Am. Chem. Soc. 2012, 134, 3542.
(12) Patra, A., Wijsboom, Y. H.; Leitus, G.; Bendikov, M. Org. Lett. 2009, 11, 1487.
(13) Mahatsekake, C.; Catel, J. M.; Andrieu, C. G.; Ebel, M.; Mollier, Y.; Tourillon, G.; Phosphorus, Sulfur Silicon Relat. Elem.: 1990; Vol. 47, p 35.
(14) Shen, Z.; Dornan, P. K.; Khan, H. A.; Woo, T. K.; Dong, V. M. J. Am. Chem. Soc. 2009, 131, 1077.
(15) (a) Kiriy, A.; Senkovskyy, V.; Sommer, M. Macromol. Rapid Commun. 2011, 32, 1503. (b) Bronstein, H. A.; Luscombe, C. K. J. Am. Chem. Soc. 2009, 131, 12894. (c) Brusso, J. L.; Lilliedal, M. R.; Holdcroft, S. Polym. Chem. 2011, 2, 175. (d) Okamoto, K.; Luscombe, C. K. Polym. Chem. 2011, 2, 2424.
(16) (a) Wu, P. T.; Ren, G. Q.; Li, C. X.; Mezzenga, R.; Jenekhe, S. A. Macromolecules 2009, 42, 2317. (b) Yu, X. H.; Yang, H.; Wu, S. P.; Geng, Y. H.; Han, Y. C. Macromolecules 2012, 45, 266. (c) Zhang, Y.; Tajima, K.; Hashimoto, K. Macromolecules 2009, 42, 7008. (d) Wu, Z. Q.; Ono, R. J.; Chen, Z.; Bielawski, C. W. J. Am. Chem. Soc. 2010, 132, 14000.
(17) (a) Locke, J. R.; McNeil, A. J. Macromolecules 2010, 43, 8709. (b) Palermo, E. F.; McNeil, A. J. Macromolecules 2012, 45, 5948.
(18) Somanathan, N.; Radhakrishnan, S.; Thelakkat, M.; Schmidt, H. W. Macromol. Mater. Eng. 2002, 287, 236.
(19) Somanathan, N.; Radhakrishnan, S. Int. J. Mod. Phys. B 2005, 19, 4645.
(20) Shen, Z.; Dornan, P. K.; Khan, H. A.; Woo, T. K.; Dong, V. M. J. Am. Chem. Soc. 2009, 131, 1077.
(21) Sweat, D. P.; Stephens, C. E. Synthesis-Stuttgart 2009, 3214.

1. A compound of formula (5): 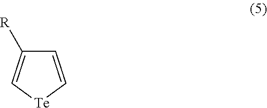
wherein R represents a monovalent organic group, or a salt thereof, wherein
when R is a linear unsubstituted alkyl, R is C2-C50 alkyl.

2. A compound of claim 1, wherein:
R is:
straight-chain or branched alkyl, optionally substituted with one or more of cycloalkyl, heteroalkyl, heterocycloalkyl, aryl, heteroaryl, nitro, carboxyl, formyl, and —C(O)—R¹ in which R¹ is alkyl, cycloalkyl, heteroalkyl, heterocycloalkyl, aryl and heteroaryl;
cycloalkyl, optionally substituted with one or more of alkyl, cycloalkyl, heteroalkyl, heterocycloalkyl, aryl, heteroaryl, nitro, carboxyl, formyl, and —C(O)—R¹ in which R¹ is alkyl, cycloalkyl, heteroalkyl, heterocycloalkyl, aryl and heteroaryl;
heteroalkyl optionally substituted with one or more of cycloalkyl, heterocycloalkyl, aryl, heteroaryl, nitro, carboxyl, formyl, and —C(O)—R¹ in which R¹ is alkyl, cycloalkyl, heteroalkyl, heterocycloalkyl, aryl and heteroaryl;
heterocycloalkyl optionally substituted with one or more of alkyl, heteroalkyl, aryl, heteroaryl, nitro, carboxyl, formyl, and —C(O)—R¹ in which R¹ is alkyl, cycloalkyl, heteroalkyl, heterocycloalkyl, aryl and heteroaryl;
aryl optionally substituted with one or more of alkyl, cycloalkyl, heteroalkyl, heterocycloalkyl, aryl, heteroaryl, nitro, carboxyl, formyl, and —C(O)—R¹ in which R¹ is alkyl, cycloalkyl, heteroalkyl, heterocycloalkyl, aryl and heteroaryl; or
heteroaryl optionally substituted with one or more of alkyl, cycloalkyl, heteroalkyl, heterocycloalkyl, aryl, heteroaryl, nitro, carboxyl, formyl, and —C(O)—R¹ in which R¹ is alkyl, cycloalkyl, heteroalkyl, heterocycloalkyl, aryl and heteroaryl.

3. A compound of claim 2, wherein R is C2-C50 alkyl.

4. A compound of formula (4): 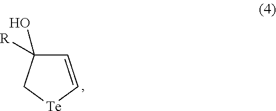
wherein R is a defined in claim 1.

5. A compound of formula (B): 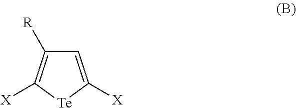
wherein:
each X is, independently of the other X, F, Cl, Br, I, H, Li, Na, MgX¹, B(OR²)₂, B(OH)₂, or SnR′″₃,
X¹ is Cl or Br,
R² for B(OR²)₂ is C1-C10 alkyl or optionally substituted C1-C10 alkylene bridging the oxygen atoms bound to B,
each R′″₃ is C1-C10 alkyl and are the same or different from each other, and
if one X is H, then the other X is not H, and
R is as defined in claim 1.

6. An oligomeric or polymeric compound comprising two or more tellurophene-2,5-diyl groups covalently linked to each other at one or the other of the 2- and 5-positions of each tellurophene ring, wherein each of the tellurophene rings is substituted at the 3- or 4-position thereof.

7. A compound of claim 6, wherein said compound is of formula (A): 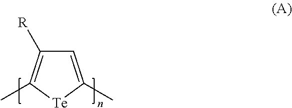
wherein n is an integer greater than 1; and
each R is, independently of each other R, as defined by claim 1.

8. A compound of claim 7, wherein the value of n is between 10 and 200.

9. A compound of claim 8, wherein the value of n is between 30 and 180.

10. A compound of claim 6, wherein the value of n is greater than 10 and the compound has a regioregularity of at least 50%.

11. (canceled)

12. (canceled)

13. (canceled)

14. (canceled)

15. A conjugated polymer comprising a compound as defined by claim 6, wherein the polymer has a number average molecular weight (Mₙ) that is at least 2,000 when measured by gel permeation chromatography relative to polystyrene standards.

16. (canceled)

17. (canceled)

18. (canceled)

19. A polymer according to claim 15, wherein the polymer is a homopolymer.

20. A polymer according to claim 19, comprising the compound of formula (A) in which R is C1-C20 alkyl.

21. A film comprising a polymer as defined by claim 15.

22. A film of claim 21, having a thickness of between 1 and 10,000 nm.

23. (canceled)

24. A composite material comprising a polymer layer comprising a polymer as defined by claim 15, and a support disposed on at least one side of the polymer layer.

25. An optoelectronic device comprising the composite material of claim 24.

26. The device of claim 25, wherein the device is a diode, a light-emitting diode, a transistor, a solar cell, a photodiode or a light-emitting transistor.

27. An electrode installed in contact with a film as defined by claim 21.

28. A solar cell comprising the electrode of claim 27.

29. A semiconductor composite material comprising a polymer as defined by claim 15 in combination with an electron acceptor material.

30. A method of preparing a polytellurophene compound, the method comprising exposing a compound of formula (5) to an electrochemical potential of from 0.1 to 3.0 V for a period of time sufficient to form the compound, wherein the R of formula (5) is as defined in claim 1.

31. A method for preparing a polytellurophene compound, the method comprising:
(i) activating a monomer of formula (B) at the 2- and 5-positions of the tellurophene ring; and
(ii) coupling the activated monomer in the presence of a coordination catalyst,
wherein formula (B) is as defined in claim 5.

32-39. (canceled)
